(19) United States
(12) Patent Application Publication
(10) Pub. No.: US 2005/0033594 A1
(43) Pub. Date: Feb. 10, 2005

(54) Flow management analysis system and method for healthcare related business

(76) Inventor: Michael Y. Shen, Fort Lauderdale, FL (US)

Correspondence Address: CAHN & SAMUELS LLP, 2000 P STREET NW, SUITE 200, WASHINGTON, DC, 20036, US

(21) Appl. No.: 10/858,396

(22) Filed: Jun. 2, 2004

Related U.S. Application Data
(60) Provisional application No. 60/474,609, filed on Jun. 2, 2003.

(51) Int. Cl. G06F 17/60

(52) U.S. Cl. 705/1; 705/2

(57) ABSTRACT

The present invention provides a method of analyzing a business flow process within an healthcare organization comprising. One or more working units are mapped within the organization. A relationship between the one or more working units is mapped. Data is collected from the one or more working units. A flow path is mapped through the working units for the business flow process. Flow indexes are computed and/or recorded corresponding to the one or more working units based on the data collected from the one or more working units. A flow process analysis is performed on the workflow based at least in part on the flow indexes.

This application claims the benefit of Application Ser. No. 60/474,609 filed Jun. 2, 2003 which is herein incorporated by reference in its entirety.

### I. FIELD OF THE INVENTION

This invention relates to the field of business flow management and more particularly, to methods and systems for engineering structures and business flows, for analyzing performance of business flows, and optimizing flow processes in the healthcare field.

### II. BACKGROUND OF THE INVENTION

Objective assessment, monitoring and optimizing of business processes is very important to all businesses, especially those related to healthcare. Business flows in healthcare organizations are far more complex than those of non-healthcare organizations.

Healthcare organizations frequently comprise a plurality of often fragmented business activities. Each of these business activities generate data relating to their specific function(s). This data is often stored locally at the business activity. Heretofore, the data generated by business activities has remained fragmented and unmined. The present inventor has determined that data generated by the business activities may be collected and organized to perform macroscopic analysis of business flows within the organization and thus facilitate optimization of the business flows, identification of obstacles in the business flows, and quantification of the business flows.

Notwithstanding the usefulness of the above-described methods, a need still exists for

### III. SUMMARY OF THE INVENTION

The present invention provides a method of analyzing a business flow process within an healthcare organization comprising. One or more working units are mapped within the organization. A relationship between the one or more working units is mapped. Data is collected from the one or more working units. A flow path is mapped through the working units for the business flow process. Flow indexes are computed and/or recorded corresponding to the one or more working units based on the data collected from the one or more working units. A flow process analysis is performed on the workflow based at least in part on the flow indexes.

### IV. BRIEF DESCRIPTION OF THE DRAWINGS

FIG. 1 shows a chart of a flow management system.

FIG. 2 depicts a structure map of working units in accordance with the invention and their network components.

FIG. 3 is a grid showing various types of flows and their major steps.

FIG. 4 is a flow diagram in accordance with the invention.

FIGS. 5a and 5b show a finance flow process grid and a finance life cycle grid, respectively.

FIGS. 6a and 6b illustrate a technology flow process grid and a technology life cycle process grid, respectively.

FIGS. 7a and 7b depicts an information flow process grid and an information life cycle grid, respectively.

FIG. 8 depicts a reconstructed flow diagram in accordance with the invention.

FIG. 9 is a block diagram of an input-output analysis.

### V. DETAILED DESCRIPTION OF THE DRAWINGS

The present invention provides a comprehensive system and method for analyzing and optimizing business flow processes in a healthcare organization. As used herein, the term healthcare organization refers to any healthcare related business including healthcare providers, such as hospitals, clinics, homecare, individual physician or group practice; healthcare insurance providers, such as insurance companies; healthcare related production businesses, such as pharmaceutical companies, equipment or device manufacturing companies; consulting companies, and professional or government organizations, etc. The present application describes the invention using a hospital as an exemplary healthcare organization.

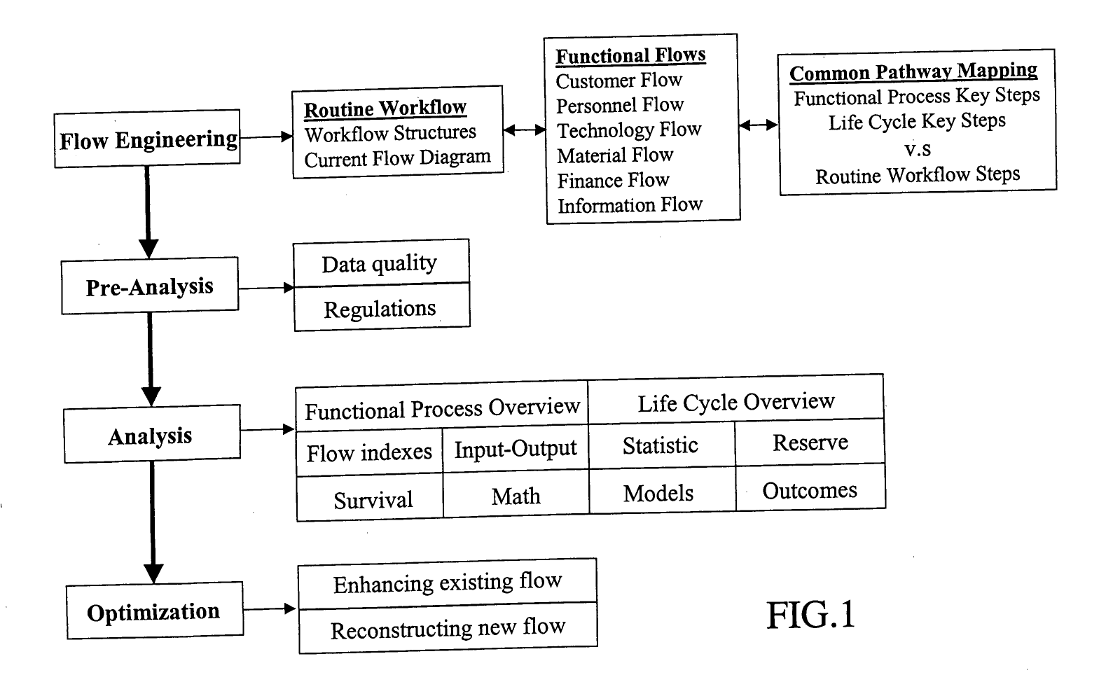

An overview of the flow management system and method of the present invention is depicted in FIG. 1. As illustrated, there are four major components in the diagram, engineering, pre-analysis, analysis and optimization. Any one or combination of these components may be claimed as an invention herein.

Engineering

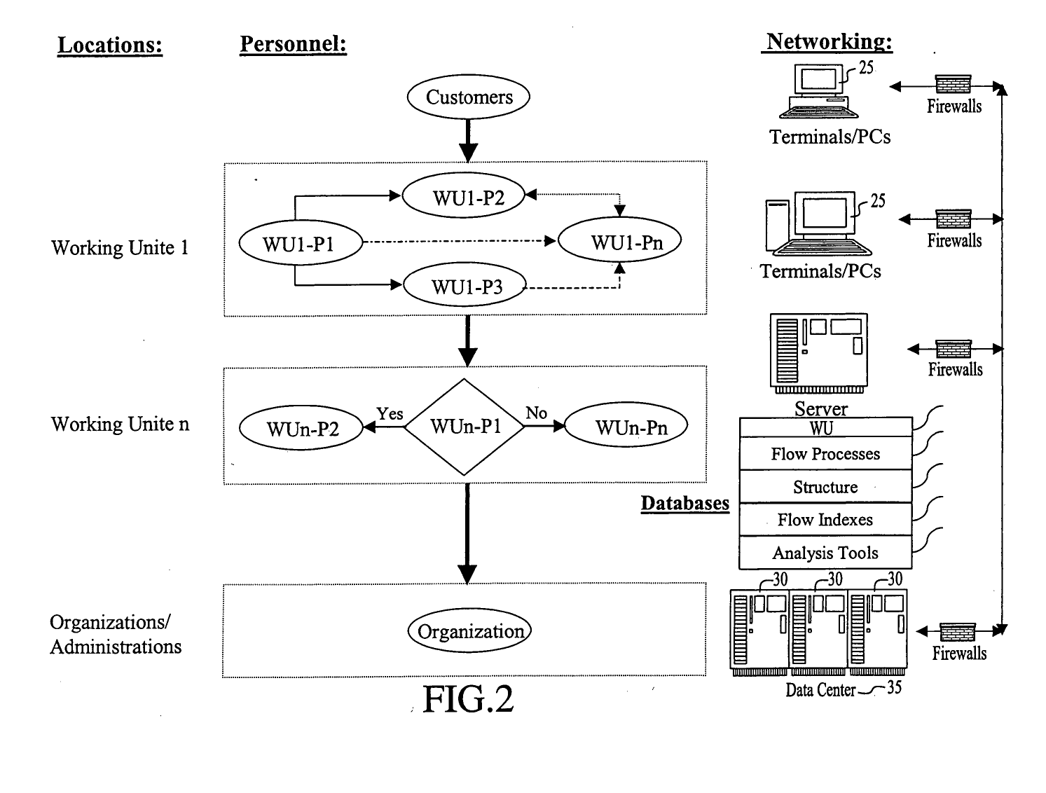

By way of general explanation, referring to the flow engineering component of FIG. 1, it is desirable to map existing structures of the organization into working units (WUs). As used herein, the term working unit refers to a business activity of the organization such as a department, division, personnel or some other subunit of the organization that performs one or more functions. Depending upon the structure of the organization as shown in FIG. 2, working units 20 may be subdivided into an infinite number of parts, e.g., sub-working units sub-sub-working units, etc. Typically, each working unit 20 includes personnel (not shown), computing devices 25 and other equipment (not shown), e.g. diagnostic equipment, medical instruments, tools and supplies. To facilitate data gathering, computing devices 25 are preferably connected to a data center 30 including one or more servers 35. The working units may generate data in various formats from a variety of operating systems and programs. Data center 30 extrapolates and converts data to a usable format.

Upon establishment of the WUs, the relationship among and between WUs is mapped. More particularly, the sequence and relationship of the WUs may be mapped as well as their hierarchy. Once the WUs are mapped, flow diagrams may be created for each routine workflow process. As used herein, the term routine workflow refers to current workflow at given WU or organization.

Analysis of routine workflow facilitates objective quantification of simple customer related parameters such as volume of patients and the speed of evaluation of the patients, etc. An exemplary routine workflow for a patient who comes to a cardiology clinic comprises the steps of 1) registration, 2) nurse evaluation and 3) cardiology consultation. Although useful, routine workflow analysis does not provide information of performance parameters such as accuracy (comparing treatment regiment to standard of care of Clinical Practice Guidelines), efficiency of initial diagnosis, appropriate testing selection for diagnosis, patient management, and clinical outcomes.

To objectively quantify performance of an organization, a unique standard format is preferably developed to classify functional flow processes. Different from routine workflow processes, functional flow processes are directed to the key steps of performance on different aspects of the organization's business. In keeping with the invention, functional flow processes may be classified into several categories including but not limited to customer flow, personnel flow, technology flow, material flow, finance flow and information flow. Once classified, the major functional steps for each flow in addition to their routine workflow may be identified or mapped. Although the flow diagram might be different for each organization or WU, the functional common pathway is quite similar for any business activity. Mapping out the key steps can standardize the WU functionality for digitization and quantitative analysis. In addition, major steps in the life cycle of each flow are preferably identified.

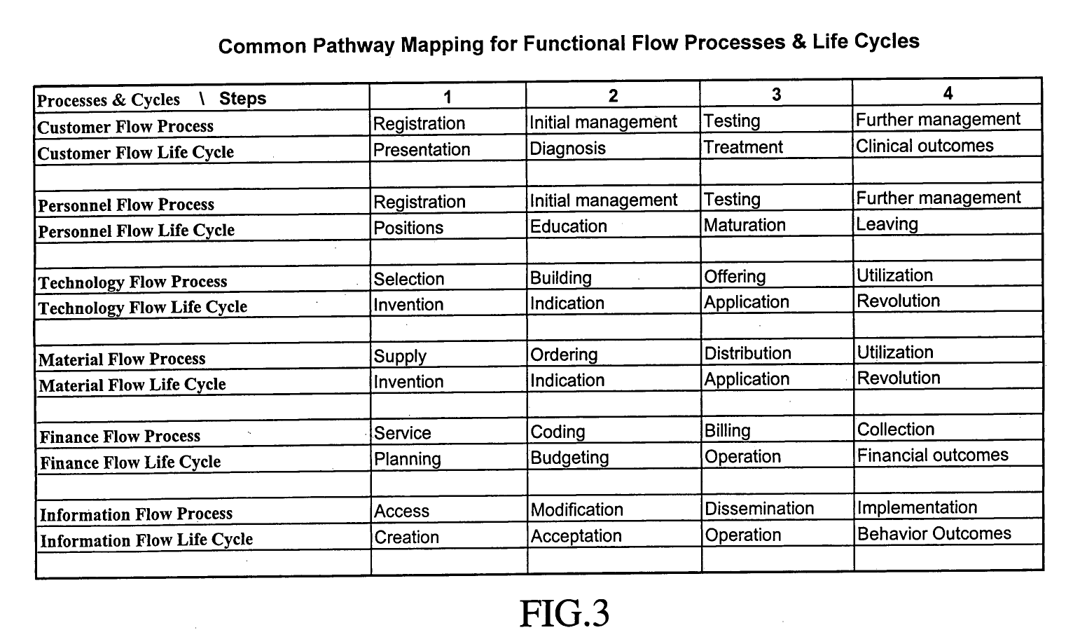

Turning more specifically to the functional flows, the customer flow process maps the specific steps of a customer going through the WU's. The major steps of various functional flows are depicted in grid format in FIG. 3. The customer flow process preferably includes four steps: 1) registration, 2) initial management, 3) testing and 4) further management. Registration includes registering into the service system of the organization and being approved by the organization or insurance for responsibilities and finance. Initial management includes initial assessment and treatment by RNs, MDs or other medical professionals. The testing step includes diagnostic testing such as imaging. The further management step includes further assessment and treatment that can be the same or different from initial management. This step is preferably offered based on testing results or patient response.

The customer flow life cycle preferably includes four major components, 1) presentation, 2) diagnosis, 3) treatment and 4) clinical outcomes. The presentation component includes symptoms or signs of disease. The diagnosis component defines diseases including their nature, stage and prognosis. The treatment component provides ways to cure or control diagnosed illness, including use of medications, surgery, complementary medicine methods, therapies, etc. The clinical outcomes component represents the results of the treatment based on diagnosis.

The Personnel flow process preferably characterizes coordination and distribution of work, such as scheduling all organization personnel in variety of capacities, shifts, locations, and WUs to provide service in a business process.

As shown in FIG. 3 the personnel flow process, like the customer flow process, preferably includes four steps: 1) registration, 2) initial management, 3) testing and 4) further management. Administrative personnel attend to the registration step, RNs and MDs attend to the initial management step, testing may be performed at pathology and radiology labs and either the initial MD or specialty MD provides further management. The personnel flow process may also inform scheduling, rotation or coverage at the healthcare organization.

The life cycle of the personnel flow is preferably divided into four major components: 1) positions, 2) education, 3) maturation and 4) leaving. Positions refers to initial hiring or assuming a specific role of a task. Education refers to training and the learning process, including continuing education. Maturation refers to the level or job status reached. Leaving refers to employment status, e.g., leaving the position or the role, including retirement or firing.

The technology flow process preferably characterizes equipment, technical tools and machinery of the organization, such as, e.g., computers, CT scans, and software packages. The technology flow process preferably includes four steps: 1) selection, 2) building, 3) offering and 4) utilization. Selection refers to selecting an adequate technology for use within the WU or the organization. Building refers to building a local service. Offering refers to offering specific services. Utilization refers to the use of the technology.

The technology flow life cycle is preferably divided into four major components: 1) invention, 2) indication, 3) application and 4) revolution. Invention refers to invention of a new technology. Indication refers to the extent to which a technology is designed for use for a specific task. Application refers to actual use for certain patients or purposes. Revolution refers to technology improvements and changes.

The material flow process preferably characterizes supplies of the healthcare organization, such as films for radiology, papers for office, needles for a clinic, medications for doctors and pharmacists, etc. The material flow process is preferably divided into four steps: 1) supply, 2) ordering, 3) distribution and 4) utilization. Supply refers to the supply resources for materials that can be analyzed in terms quality, economy and convenience. Ordering refers to the act of purchasing. Distribution refers to distribution issues, such as storage, transportation and delivery. Utilization refers to the utilization of materials.

The material flow life cycle is preferably divided into 4 major components: 1) invention, indication, 3) application, and 4) revolution. Invention refers to inventing a new material, including medications, etc. Indication refers to the extent to which a material is designed for use for a specific task. Application refers to actual use for certain patients or purposes. Revolution refers to a material improvements and changes.

The finance flow process preferably characterizes activities and circulations related to finance and preferably includes four steps: 1) service, 2) coding, 3) billing and 4) collection. Service refers to seeing a consultation, such as a doctor. Coding: refers to the purchase level coded for charge or reimbursement. Billing refers to the charge input into a system or delivered to the payor. Collection refers to obtaining the money for the services rendered.

The finance flow life cycle is preferably divided into four major components including 1) planning, 2) budgeting, 3) operation and 4) financial outcomes. Planning: refers to setting goals. Budgeting refers to provision of funds. Operation refers to execution of financial activities, including business activities. Financial outcomes refers to cost sand benefits, both financial and otherwise.

The information flow process preferably characterizes information transformation process, including knowledge, regulations, standards and new updates, etc. The information flow process is preferably divided into four steps: 1) access, 2) modification, 3) dissemination and 4) implementation. Access refers to obtaining the information. Modification refers to tailoring the use to suit the WU or organization. Dissemination refers to where, when and to whom information may flow. Implementation refers to the use of the information for action.

The information flow life cycle is preferably divided into four major components, 1) creation refers to new knowledge, information creation. Acceptation refers to the level of acceptance by users. Operation refers to providing and using the information. Behavior outcomes refers to the impact of the information on people's action.

Pre-Analysis

With respect to the pre-analysis component, prior to the data center collecting data from the working units, it is desirable to verify the quality of the data. This may be done with a data verification routine or by manually checking data for accuracy and reliability. As an exemplary measure of accuracy, data may be scored according to its level of accuracy. For example on a 1-5 scale, poor quality data may be given a score of 1, high quality data may be given a score of 5 while intermediate or neutral data may be given a score of 3. All data may be recorded in one or more working unit databases 50 of FIG. 2 and data having a score less than 3 should be annotated with an indication of its quality. Data stability (the mean score and the standard deviation of the score) and validity (the difference between input data and real data) may be assessed over time.

Analysis

Turning to the analysis component, multiple tools may be employed to quantify and display information related to routine and functional workflow processes. The present invention includes a routine workflow analysis engine, a process analysis engine and life cycle analysis engine. Each of the analysis engines preferably comprise program code resident on one or more servers 35. Each of the analysis engines preferably employs flow indexes to assist in the flow analysis process. Flow indexes may be computed or recorded any one or more of the analysis engines or by a separate flow index calculator. As used herein the term flow index refers to a variable that characterizes flow. These variables may comprise WU data or they may be computed from WU data. Flow indexes may be quantitative, e.g., 0-100%; semi-quantitative, e.g., scale from 1-5; or descriptive, e.g., high, medium or low.

The present invention employs several flow indexes including: 1) volume, 2) time and speed, 3) types or classes, 4) quality and accuracy, 5) congestion/waiting time, 6) overflow and loss and 7) outcomes. Volume refers to the number of elements, such as the number of customers or pieces of information. Time and speed refers to the time and rate of elements passing through a WU such as the time of 50% of information adopted by a division, referred to as half-life of information transformation. Types or classes refer to the type or kind of flow elements such as patients with severe congestive heart failure vs. patients only with hypertension. Quality and accuracy such as % of satisfied patients or % of patients completing surgery without complications. Congestion/waiting time refers to the waiting period of the flow elements before passing through a WU, such as catheters being stored on a shelf for 7 months prior to usage. Overflow and loss refers to flow elements overflowed and/or lost passing through a WU, such as outdated and wasted medications, patients waiting too long to receive service from a first diagnostic center and, as a result, seeking service from a second diagnostic center. Outcomes refer to the final results of different aspects of business flow relating to quantity and quality. One exemplary outcome category is clinical outcomes such as patient's death, stroke, myocardial infarction and other complications. Another exemplary outcome category is diagnostic outcomes, e.g. sensitivity and specificity. Still another exemplary outcome category is treatment outcomes, e.g., survival rate. Other exemplary outcome categories include utilization outcomes (such as the number of patients who had PTCA or bypass surgery), financial outcomes (cost and benefits), service outcomes (satisfaction rate), compliance outcome (% meeting standards). The foregoing flow indexes are exemplary and not exhaustive. Additional flow indexes may be created based on the purpose of evaluation or imported from other systems.

In addition to the flow indexes, other analytical parameters are may be computed in connection with both process and life cycle analyses. These analytical parameters are preferably computed by one or more parameter calculation engines which preferably reside on the computing devices 35 of data center 30

Input and Output

The input and output analysis is the basic analytic tool for the function of a WU relative to the flow going through the WU, such as customer flow to a division and information flow to personnel. For example, the inputs of personnel flow to a division include the number, type and capacity of a group of employees and the outputs include scheduling, work distribution and coordination of a division. In a further example, the inputs of information flow to an individual include updates of knowledge in certain technology or regulations and the outputs include adoptions or implementation by the individual of the technology or regulation. The ultimate flow analysis will be related to customer flow, such as in the last two examples, the impact of the personnel flow in a division or the information flow in an individual on customer flow for its quality and quantity, etc.

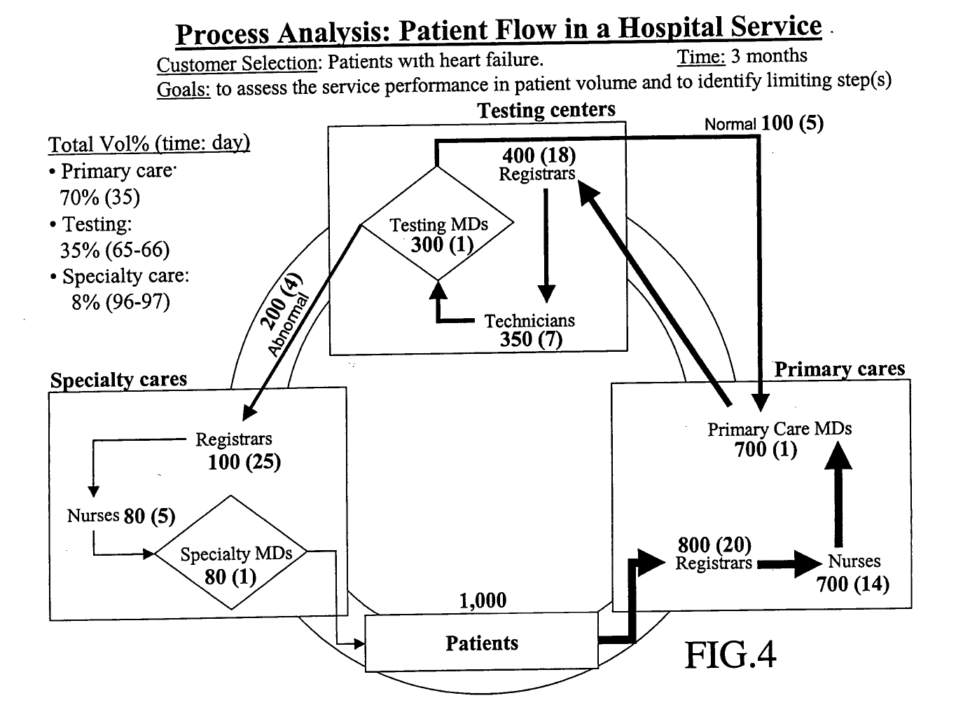

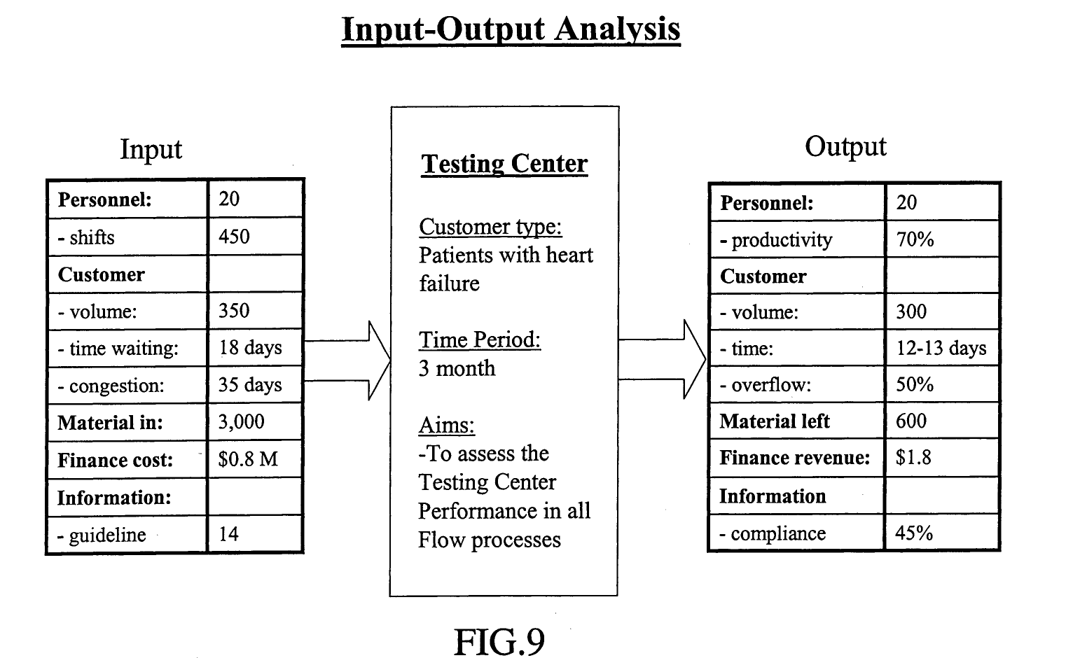

FIG. 9 illustrates an exemplary input and output analysis for the testing center of FIG. 4. It comprehensively analyzes the performance of all of the flows and points out the problems at the testing center. For example, the input patient volume was 350 and the output volume was 300. This suggests that 50 patients did not have their tests done. The reasons for these patients absence need to be investigated. The waiting time for accessing the testing center was 18 days and the time to get the test results was additional 12-13 days. These analyses will help the organization to quantify performance and identify limiting steps in different aspects of the service.

Statistical Analysis for Performance Distribution

Flow statistical distribution analysis uses statistical tools to quantify performance of each WU in an organization or individuals in a WU. Mean and median are the common indexes to quantify overall performance of flow(s), such as mean volume of patients and median time to transform a national practice guideline (standard information) to a WU. Standard Deviation and Standard Error are the indexes used to quantify the degree of variations of individuals in a group from mean. For example, the number of standard deviations (or Sigma) and cutoff can be used to quantify the outliers to further define quality or performance.

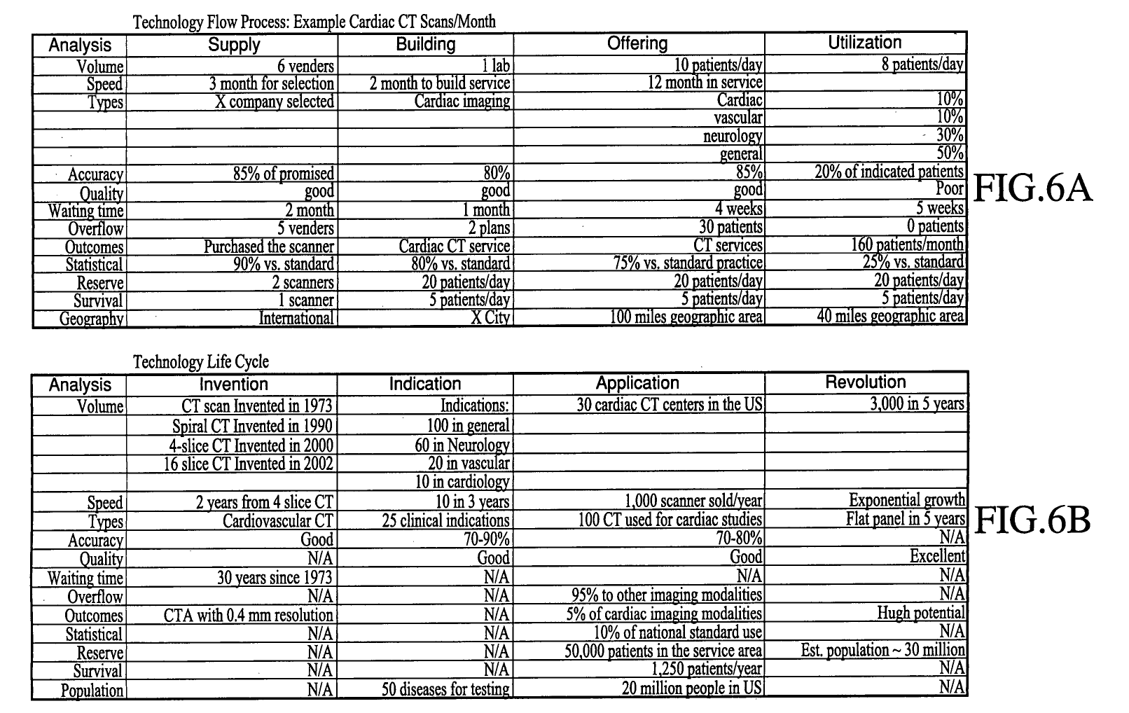

FIG. 6 illustrates an exemplary statistical curve showing the performance of Personnel X was within 95% of the WU. It seems quite reasonable. Actually, if the working defect is calculated per million, the defects were actually over 300,000. However, if the statistical probability improved to 99.99966%, the defect per million would decrease to 3. The improvement was 20,000 times.

Outcomes: Comparison with Standards or Benchmarks

Outcome analysis is a cross section analysis looking at the end results or certain points of a process. These results can be compared with standards to identify the difference between a working structure or an individual and related benchmarks. Benchmarks include National Standards, Practice Guidelines, etc are being entered and used for comparison to identify current status of quality and quantity of service. Organizational or WU administration or even individuals can input new service goals based on market potential or demands, including but not limited to epidemiological studies, competitions, geographic distributions, advertisement campaigns, etc. These new goals may be lower or higher than national standards.

Reserve Analysis: Maximal Stress Test

The performances of the WUs may be assessed not only against the baseline of the operation or service, but more importantly, against the ability to tolerate variations, turbulence or even disasters of internal and external environment changes, such as internal strike, or external economic downturns. The ability to handle extra requirements relative to routine baseline is called reserve, defined as the ratio of maximum performance to baseline performance. The reserve can be used to quantify an organization's or WU's ability to handle extra requirements and challenges to unexpected events. The test to investigate the reserve is called stress test.

Baseline Performance:

The relative equilibrium status of performance needs to be calculated as a baseline. This figure can be further studied in terms of percentage of mechanical or fatigue allowance. For example, stable daily performance for an individual worker can be defined as 80% of fatigue allowance.

Stress Test for Estimating Maximal and Minimal Reserve

Stress test can be performed using actual testing, such as using higher volume in a short period of time (such as a day or a week), or using computer simulations. The stress test can be applied to all flow processes, such as personnel reserve to see the maximal performance of a unit with optimizing scheduling and coordination. The ultimate goal of all flow reserve testing is to study the customer flow reserve, to test the maximal ability to handle growth over a period of time. These measurements are important for an organization with strong compatibility in the market place.

Prediction and Preparation of External Stress and Internal Growth:

Prediction of external and internal environment changes and preparation for the changes is a challenging task for an organization. This can be a part of marketing department tasks to estimate seasonal variations, economic up and downs, market changes, such as related new technology development or customer demand as well as wars, epidemic diseases, etc. Prediction of these changes can correlate to the changes of flows for an organization so that the organization can modify and prepare the reserve for these challenges.

Survival Analysis: Minimal Viability Test

Survival analysis is intended to estimate the minimal requirements for a organization's existence. It is usually done by computer simulation or estimation. It assesses the minimal flows to maintain processes alive, such as minimal financial, material, personnel, and customer flows. Figure (Table 2) illustrates the reserve and survival assessment for the testing center in the example of Figure (old 4). The maximal reserve and minimal survival are computed relative to the baseline in all flow processes.

Mathematical Analysis

Using variety mathematical methods, one can further analyze the complexities of different flows in healthcare and the interactions among the flows. For example, using Multivariable Analysis and Factor Analysis, one can assess the importance and the impact of multiple factors (performance of key steps) on patient clinical outcomes or financial outcomes, and which factor is more critical among the key steps involved in the process.

Routine workflow analysis addresses flows at the WU level. Routine workflow analysis is useful to help organizations understand functionality within current workflow paths. FIG. 4 illustrates an exemplary flow diagram of customer flow in a hospital over a three (3) month period for patients with heart failure. The diagram depicts WUs 100 and sub WUs 120. The flow diagram is preferably realized as a graphical user interface (GUI) having several dynamic elements. The direction of customer flow is illustrated by the direction of arrows within and between WUs. The magnitude of the volume of customer flow may be represented by the thickness of the arrows. Flow indexes may also be displayed on the flow diagram as either static or dynamic elements. For example, FIG. 4 illustrates the patient volume for each sub WU and the number of days elapsed for the patients to move from sub WU to sub WU.

The flow diagram is preferably generated by the routine workflow analysis engine. To assess the patient volume and flow, the routine workflow analysis engine preferably calculates the volume and time for each WU 100. The patient volume at WU 100 was 1,000, quite good for the hospital in the 3 months period. However, the volume for the testing center (WU 120) was decreased to 35% (patients actually had tests) and the volume in the specialty care unit (WU 120) was even further decreased to 8%. Through the process analysis on time, one can see the waiting period was too long, taking over 60 days to have tests performed and over 90 days to see a specialty physician from the time of making an appointment at the Primary Care Facility. As is also evident, many patients avoided or left WUs for other hospitals before reaching a testing center or before reaching a specialty care unit (overflow).

Process analysis utilizes the flow indexes quantitatively to reveal the relative value and function of each WU or subunit thereof as well as the limiting factors of the WU's and their corresponding subunits. The indexes may be normalized to the total volume of the WU to quantify a percentage of each WU to the total workflow through the WU. In addition, the flow indexes may be converted to absolute dollars to assess revenue changes with a WU.

Life cycle analysis is useful in investigating the life cycle of flows longitudinally over time. Life cycle analysis may help to assess functional stages of a given flow (early phase or mature phase) and the different stages for each WU or subunit (such as one can be at an early stage of learning and another one can be at an optimum stage). This may help to explain differences among WUs or personnel in their performance.

The difference between process analysis and cycle analysis is that the former focuses on flow going through each step of structures in a process in space, and the latte focuses on each stage of the flow in its life cycle in time. This analysis can help to assess the step or the status of flow in business processes to better understand process analysis.

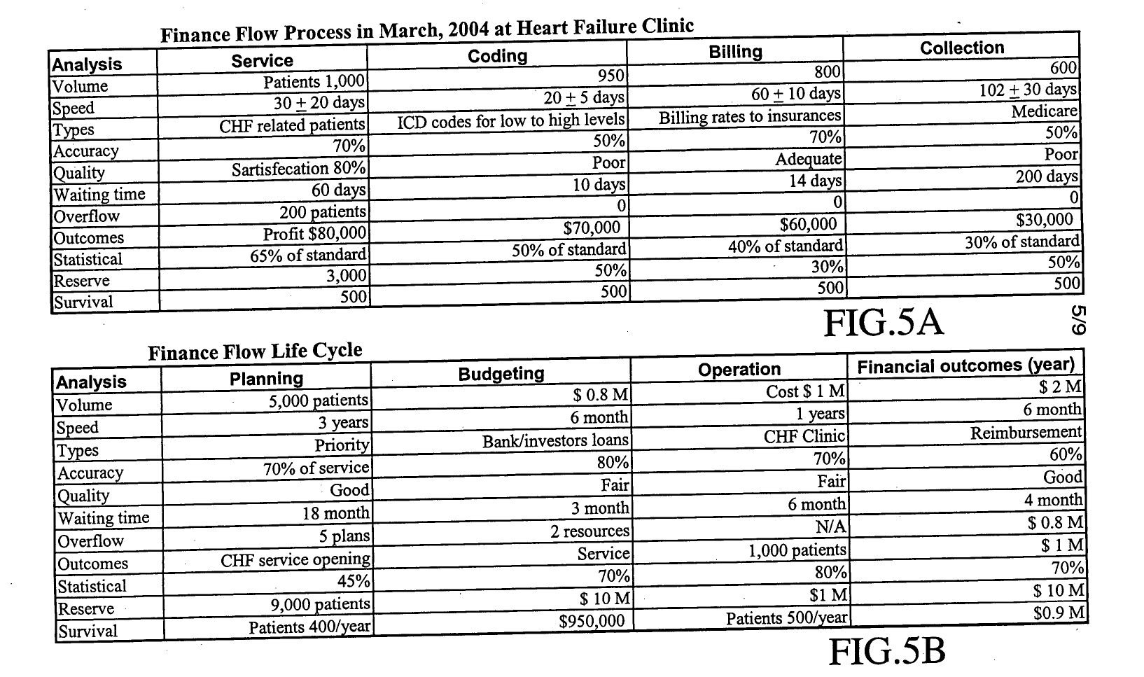

Together, process analysis and life cycle analysis help the organization understand its functionality for key steps during the performance of the flow and during the life cycle of the flow. FIGS. 5a and 5b depict exemplary process and life cycle grids respectively for finance flow over one month in a heart failure clinic. The flow indexes are positioned in the left most column of the grid under the “Analysis” heading. Immediately to the right of the “Analysis” column, the major steps for finance flow, service, coding, billing and collection are positioned along the first row.

The process grid of FIG. 5a illustrates a comprehensive quantitative performance of the finance flow process. As specified in FIG. 5a, flows may be analyzed according to flow indexes, e.g., volume, speed, etc., or according to other analytic parameters. In the example depicted in FIG. 5a by reviewing the process grid, a medical consultant can determine the following information. The clinic served 1000 patients during the month but its coding and billing were not complete on time. Along with inadequate reimbursement, the collection rate was quite low. The speeds of each steps of the process were quite slow, especially the collection time. The accuracies of billing code (ICD codes) and the level of service were quite low, which contributed to the low number of collection. The quality of billing and collection were poor. The waiting times for patients were too long and many patients left for other services. Using 6 sigma statistical analysis against the national standard, the performance of each step were low, especially the billing. However, the clinic's financial performance is above the survival baseline.

The life cycle grid FIG. 5b depicts a comprehensive quantitative performance of the finance life cycle from including the major steps of planning, budgeting, operation and financial outcomes.

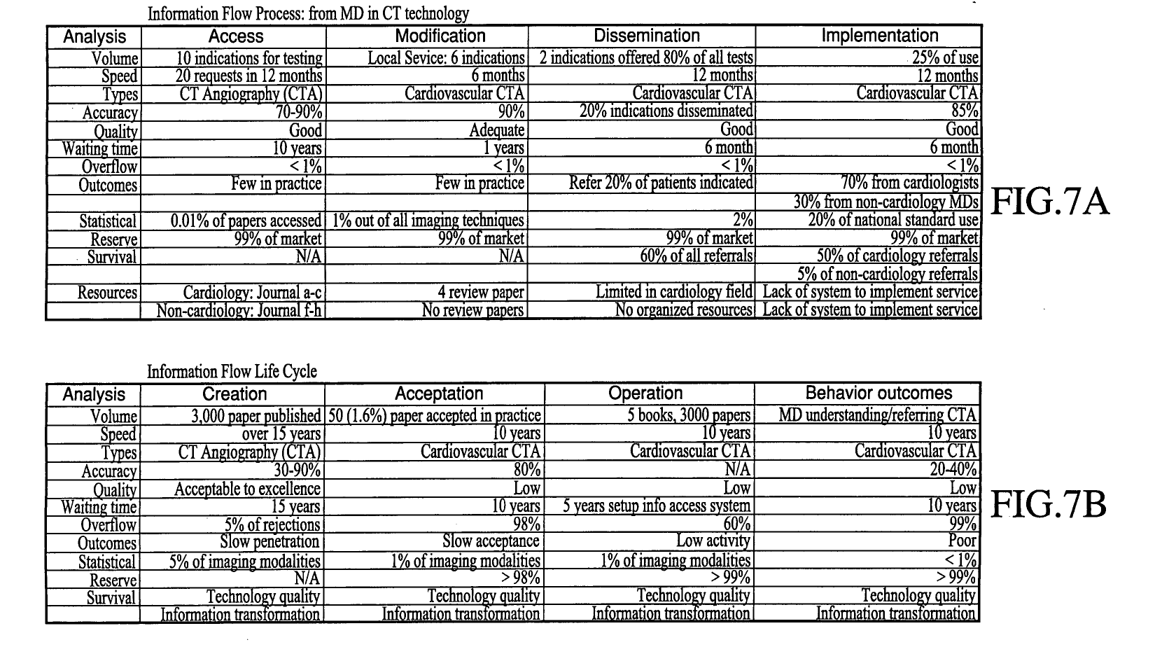

In accordance with another example, an analysis of the first 12 months of technology use for cardiac CT and the impact of information on the utilization of the new technology is examined. FIGS. 6a, 6b and 7a, 7b represent process and life cycle grids, respectively, of a technology flow process for cardiac CT scans over a one-month period and the impact of information on the utilization of new technology. The process grid of FIG. 6a depicts the quantitative state of the technology flow process. From the technology flow process grid, a medical consultant can determine, for example, that the technology supply is good with a variety of options (6 vendors) and that the technology has reached 85% of promised function at delivery from the vendor.

The technology flow process grid further reveals that the service building process is also quite good, reached 80% of standard service in the US with estimated volume 4 times that necessary to break even (20 vs. 5 patients/day in Reserve and Survival categories).

The technology flow process grid still further reveals that the offering service step has some problems. The quality of service is good, and it reached 75% of standard and a variety of areas of service in cardiac, vascular and neurology. Yet the scheduled volume/day only achieved 50% (10 patients/day vs. planned 20/day). And the scheduling waiting period is long for 4 weeks and 30 patients could not wait and left for other services.

The technology flow process grid yet further reveals that the utilization step has significant problems as well. On average, only 8 patients/day completed tests, 40% of the planned amount. There was more service in general than in cardiac and vascular. The referred patients only accounts for 30% who are indicated. The utilization only reached 25% of performance based on the national standard.

The technology life cycle grid shown in FIG. 6b reveals that invention step is excellent with exponential growth since 2000. The Indications step shows that cardiac CT studies are only few compared to other imaging modalities. The application step is only beginning with great potential. The revolution for CT most likely will be the flat panel in the cardiac imaging market within 5 years.

The information flow process grids depicted in FIG. 7a has information that assists in understanding the relationship of cardiac CT utilization with information. The information process flow grid reveals that the access step is very limited, only 20 times over a 12 month service period. Only 0.01% of all papers were accessed from the library. The modification step makes more limited use in local market. The dissemination step has major problems with only 20% of indicated patients actually referred. The implementation step results are quite poor, especially from non-cardiology MDs.

The information flow life cycle grid FIG. 7b reveals that the creation step is quite promising with many papers published in the CT area with very few rejected for publication. The acceptation step has major problems with only 50% accepted for use over 10 years. The operation step for information is also suboptimal, taking too much time to setup adequate information systems (such as special index or library) for access. The behavioral outcomes are also quite disappointing.

Through the above comprehensive, objective and quantitative interaction analysis of the grids of FIGS. 6a, 6b and 7a, 7b, a medical consultant may learn that the low utilization of cardiac CT is limited by information transformation process for more physicians to understand the indications and advantage of cardiac CT as well as local service group in education and quality service.

Another example, to assess a new information flow performance going through different parts or individuals in a division, one can use process analysis to quantify relative time and utilization of the information in the division. However, each individual may be at the different phases of the information cycle, one might be only at access, the early phase of the information flow cycle and another might be at adoption, the later and mature phase of the cycle. Integration of cycle analysis with process analysis can help one to better understand where the difference of the information going through each component of WUs is.

Optimization

The foregoing analysis provides the medical consultant with the tools to enhance existing flows and or to reconstruct existing flows. For example, the functions of workflow may be improved by changing certain aspects of the workflows such as enhancing operations, increasing coordination of certain components and functions, e.g., those depicted in FIGS. 4 and 9 with existing structures and flow diagrams.

Reconstruction of flow structures is a creative process. In reconstruction, any component, sequence or combinations may be changed as long as the flow performance is improved. For example, one can use fewer steps, different routes or new components with new technology to increase efficiency and accuracy.

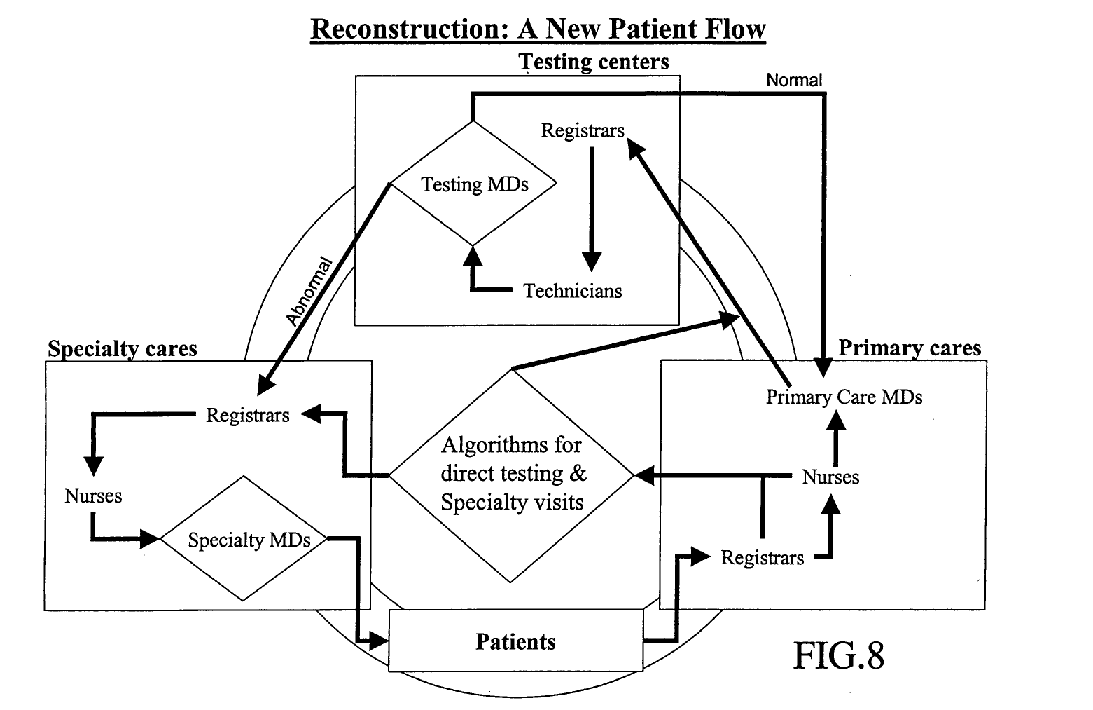

Logic algorithms may also be created with specific indications, conditions and limits, which can be entered and integrated with the overall flow system. FIG. 8 illustrates an example of reconstruction of the existing hospital patient flow from FIG. 4. A new triage algorithm is created, a set of rules (such as history of myocardial infarction, signs of heart failure with shortness of breath on exertion, etc) may identify high risk patients for early testing and direct specialty consultation (such as cardiology) instead of going through primary care physicians first. This can avoid long waiting periods, delayed patient care and patient drop offs.

The reconstruction process is preferably performed using the flow diagram to reroute, change the numbers of components or sequence and to estimate a flow performance.

Those skilled in the art will appreciate that various adaptations and modifications of the above-described preferred embodiments can be configured without departing from the scope and spirit of the invention. Therefore, it is to be understood that, within the scope of the appended claims, the invention may be practiced other than as specifically described herein.

1. A method of analyzing a business flow process within an healthcare organization comprising:
mapping one or more working units within the organization;
mapping a relationship between the one or more working units;
collecting data from the one or more working units;
mapping a flow path through the working units for the business flow process;
computing or recording flow indexes corresponding to the one or more working units based on the data collected from the one or more working units; and
performing a flow process analysis based on the flow indexes.

2. The method of claim 1 wherein a working unit includes a division of the organization.

3. The method of claim 1 wherein a working unit includes a department of the organization.
